(19) United States
(12) Patent Application Publication
(10) Pub. No.: US 2021/0121305 A1
(43) Pub. Date: Apr. 29, 2021

(54) UNITARY PROSTHETIC FOOT AND METHOD OF MANUFACTURING THE SAME

(71) Applicants: Phong Thanh Dao, Ho Chi Minh City (VN); Thang Tan Nguyen, Ho Chi Minh City (VN); Giang Hieu Le, Ho Chi Minh City (VN); Tuan Manh Bui, Ho Chi Minh City (VN)

(72) Inventors: Phong Thanh Dao, Ho Chi Minh City (VN); Thang Tan Nguyen, Ho Chi Minh City (VN); Giang Hieu Le, Ho Chi Minh City (VN); Tuan Manh Bui, Ho Chi Minh City (VN)

(73) Assignee: TON DUC THANG UNIVERSITY, HO CHI MINH (VN)

(21) Appl. No.: 16/006,862

(22) Filed: Jun. 13, 2018

(51) Int. Cl. A61F 2/66; A61F 2/50; A61F 2/80; B33Y 80/00

(52) U.S. Cl. CPC A61F 2/6607; A61F 2/5046; A61F 2/80; A61F 2002/5075; A61F 2002/6635; A61F 2002/6692; A61F 2002/5055; B33Y 80/00

(57) ABSTRACT

A prosthetic foot and method of fabricating the same is disclosed which includes a socket assembly configured to connect to a natural limb of a patient, a sideway cylindrical ankle joint having a maze-like internal structure laterally affixed to the socket assembly; a foot assembly, laterally affixed to the sideway cylindrical ankle joint, having a dorsal portion, a phalange portion, a sole portion, and a heel portion; the phalange portion having a first end connected to the dorsal portion and a second end connected to the sole portion, the sole portion having a curve shape and connected to the heel portion; and the heel portion having a spring assembly connected to said cylindrical ankle joint.

### FIELD OF THE INVENTION

The present invention relates generally to the field of medical products. More specifically, the present invention relates to a prosthetic foot.

### BACKGROUND ART

In recent years, the need to restore the form and function for people with limb loss is ever increasing. According to the statistics and calculation processes of the World Health Organization (WHO), approximately 0.5% of the population (or 400,000 peoples) have access to prosthetic care and to receive physical therapy assistances. In Vietnam, this need is pressing because of the natural disasters, traffic accidents, and land mines accidents. In addition, limb loss can be the result of trauma, malignancy, disease such as peripheral vascular disease, or congenital anomaly.

For the above reasons, many prosthetic products are conceptualized, developed, and commercialized by different health and physical therapy companies. However, the majority of the prosthetic products in the market today is commonly comprised of different components connected together with an elastic mechanism, e.g., springs. When a patient walks, the prosthetic foot is compressed and released providing a propulsion when the patient lifts and makes a next step. As a result, the walking motion is awkward and unnatural. The conventional prosthetic feet fail to satisfy the need to recover the function and form for the patients with limb loss. More particularly, the conventional prosthetic feet fail to improve the walking posture, causing side effects such as spending unnecessary energy, increasing pressures on the subtalar joint, knee joint, and the hip joint.

The conventional prosthetic limbs are made from different parts assembled together. Therefore, they fail to synchronize the musculoskeletal movements of the real feet. Even the technique disclosed by Scott Summit in U.S. Pat. No. 8,366,789 attempts to improve the performance of a prosthetic limb, only the generic surface is adjusted to assimilate to the intact limb. The end of the amputated limb is also measured to design the socket. However, the prior art Summit's prosthetic limb is still constituted of discrete gears such as circular feature 653, AC clamp 971, etc. operating together to make the walking possible. The prior art Summit's prosthetic limb is rigid, unnatural, and mechanical. In addition, Summit's prosthetic limb is expensive and assembly time is high. Prior art prosthetic foot either does not pay attention to the malleolus bone (ankle bone) or designed them very stiff. Prior or conventional designs do not pay attention to the subtalar joint or designed the subtalar joint without flexibility.

Therefore what is needed is a prosthetic limb based on the flexible mechanism and the elasticity of the composite material which absorbs vibrations avoiding the effects on the subtalar joint. Furthermore, there is a need for a prosthetic foot that stores kinetic energy in order to provide a propulsion for the next step. There are needs for structure that does not have any joints, no gaps between joints, no friction, high-precision, manufactured from a single-piece mold or from 3D printing technology that decreases costs and assembly time.

### SUMMARY OF THE INVENTION

Accordingly, an objective of the present invention is to provide a prosthetic foot that includes a socket assembly configured to connect to a natural limb of a patient, a sideway cylindrical ankle joint having a maze-like internal structure laterally affixed to the socket assembly; a foot assembly, laterally affixed to the sideway cylindrical ankle joint, having a dorsal portion, a phalange portion, a sole portion, and a heel portion; the phalange portion having a first end connected to the dorsal portion and a second end connected to the sole portion, the sole portion having an arch shape and connected to the heel portion; and the heel portion having a spring assembly connected to said cylindrical ankle joint.

Another objective of the present invention is to provide a method of fabricating a prosthetic foot including the steps of: (a) providing a mold having a socket section, a sideway cylindrical ankle joint section having a maze-like internal structure laterally affixed to the socket section; a foot section, laterally affixed to the sideway cylindrical ankle joint section, having a dorsal portion, a phalange portion, a sole portion, and a heel portion; the phalange portion having a first end connected to the dorsal portion and a second end connected to the sole portion, the sole portion having an arch shape and connected to the heel portion; and the heel portion having a spring assembly connected to the cylindrical ankle joint section; (b) filling the mold with compliant composite such as Glass Fiber Reinforced Plastic (GFRP); and (c) removing the mold to obtain the prosthetic foot in accordance to the present invention.

Another objective of the present invention is to design and to use 3D printing technology to print a single piece prosthetic foot described above.

Another objective of the present invention is to achieve the above-described prosthetic foot that is capable of absorbing vibration so as to avoid injury to the tibula bone and store energy due to exogenous force.

Another objective of the present invention is to achieve the ankle joint comprises a series of flexible springs organized into the structure similar to the malleolus bones of the ankle so as to provide flexibility and absorb shock or vibrations.

Another objective of the present invention is to achieve a prosthetic foot in which the dorsal is designed according to an asymptotic curve principle having flexible parallel cuts or patterns similar to the wings of a dragon flies;

Moreover, another objective of the present invention is to achieve the above described prosthetic foot made of a compliant composite material such as Glass Fiber Reinforced Plastic (GFRP) used having the ability to store energy and then release it to provide a propulsion force for the next step taken by a user;

In another objective, the horizontal and vertical slits enable the prosthetic foot to achieve movements analogous to the real foot in term of folding the sole, the metatarsal when an external force exerts thereupon due to the uneven ground.

The object of this invention is to provide a prosthetic foot to help limb-loss people to achieve full recovery and assimilate back into the community;

Another objective of the invention is to provide a low cost prosthetic foot manufactured from a single mold or from a 3D printer.

These and other advantages of the present invention will no doubt become obvious to those of ordinary skill in the art after having read the following detailed description of the preferred embodiments, which are illustrated in the various drawing Figures.

### BRIEF DESCRIPTION OF THE DRAWINGS

The accompanying drawings, which are incorporated in and form a part of this specification, illustrate embodiments of the invention and, together with the description, serve to explain the principles of the invention.

FIG. 1 is a schematic diagram illustrating a first implementation of a prosthetic foot in accordance with an exemplary embodiment of the present invention;

FIG. 2 is a schematic diagram illustrating a second implementation of a prosthetic foot in accordance with an exemplary embodiment of the present invention;

FIG. 3 is a schematic diagram illustrating a third implementation of a prosthetic foot in accordance with an exemplary embodiment of the present invention;

FIG. 4 is a schematic diagram illustrating a fourth implementation of a prosthetic foot in accordance with an exemplary embodiment of the present invention;

FIG. 5 is a flow chart illustrating a process of fabricating a prosthetic foot in accordance with an embodiment of the present invention;

### DETAILED DESCRIPTION OF THE INVENTION

Reference will now be made in detail to the preferred embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of ordinary skill in the art that the present invention may be practiced without these specific details. In other instances, well-known methods, procedures, components, and circuits have not been described in detail so as not to unnecessarily obscure aspects of the present invention.

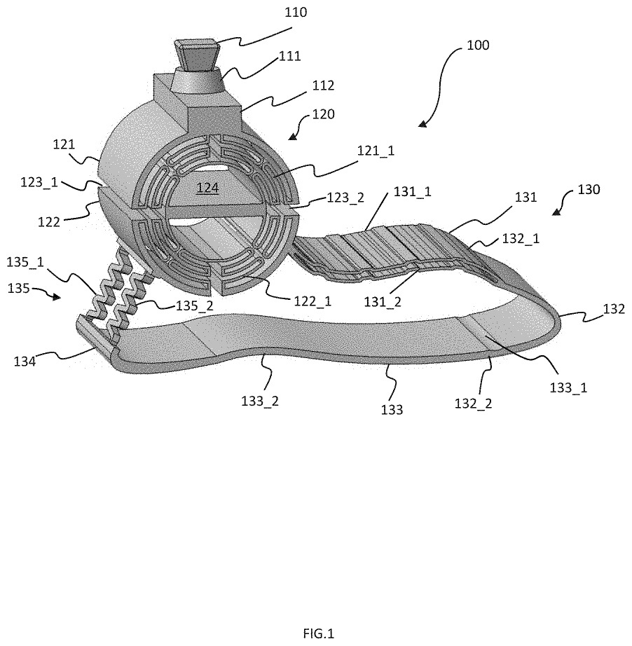

Referring now to FIG. 1 which illustrates a schematic diagram of a prosthetic foot 100 in accordance with an exemplary embodiment of the present invention. In one implementation, prosthetic foot 100 includes a socket assembly 110 configured to connect to a natural limb of a patient (not shown), a sideway cylindrical ankle joint 120 having a maze-like internal structure laterally affixed to socket assembly 110, a foot assembly 130 laterally affixed to sideway cylindrical ankle joint 120. In many implementations of the present invention, foot assembly 130 further comprises a dorsal portion 131, a phalange (or toe) portion 132, a sole portion 133, a heel portion 134, and a back heel portion 135. In accordance with a more detailed aspect of the present invention, phalange (or toe) portion 132 has a first end 132_1 connected to dorsal portion 131 and a second end 132_2 connected to sole portion 133. The connection between second end 132_2 of phalange portion 132 is separated by a groove 133_1 arranged in the direction across from the toes.

Continuing with FIG. 1, sole portion 133 has an arch shape 133_2 that matches the shape of the human sole and is connected to heel portion 134. In one exemplary embodiment of the present invention, back heel portion 135 comprises a pair of spring assembly 1351 and 135_2 connected to sideway cylindrical ankle joint 120. In accordance to the present invention, socket assembly 110, sideway cylindrical ankle joint 120, foot assembly 130, dorsal portion 131, phalange portion 132, sole portion 133, heel portion 134, and back heel portion 135 are fabricated as an integral structure from a single mold. Alternatively, the entire prosthetic foot 100 is designed and printed from a 3D printer. In both implementations, they are made of a compliant composite such as Glass Fiber Reinforced Plastic (GFRP).

In some implementations of the present invention, dorsal portion 131 has an arch asymptotic to the shape of the metatarsal bone. Dorsal portion 131 further comprises an upper membrane 131_3 and a lower membrane 1312, each having a plurality of vein patterns forming a plurality of cells similar to a dragonfly's wing pattern.

Socket assembly 110 is consisted of a connector 111 connected to a base portion 112. In one exemplary embodiment, connector 111 has a shape of an inverted truncated pyramid and base portion 112 has a shape of a truncated cone shape. Other shapes and structures of connector 111 and base portion 112 are within the scope and therefore made obvious by the present invention.

Continuing with FIG. 1, in one exemplary embodiment of the present invention, sideway cylindrical ankle joint 120 has a circular surface area further divided into an upper half 121 and a lower half 122. Upper half 121 further comprises an extension 112 laterally connected to base portion 111. In more detailed aspects of the present invention, sideway cylindrical ankle joint 120 further comprises a divider 124, spanning horizontally through a center of sideway cylindrical ankle joint 120, dividing upper half 121 from lower half 122. Divider 124 comprises a rectangular parallelepiped having a length shorter than a diameter of sideway cylindrical ankle joint 120 so as to create a first gap 123_1 and a second gap 1232 at a left side and a right side of sideway cylindrical ankle joint 120 respectively. In more detailed aspects of the present invention, the interior of upper half 121 further comprises a first plurality of sections 121_1. Each section 121_1 is radially arranged and further comprises a first zigzag pattern. Lower half 122 is laterally connected to dorsal portion 131 and heel portion 134. Similarly, the interior of lower half 122 further comprises a second plurality of sections 122_1. Each section 1221 is radially arranged and further comprises a second zigzag pattern. In one implementation, first plurality of sections 121_1 further comprises four sections; and second plurality of sections 122_1 also comprises four sections.

As shown in FIG. 1 first zigzag pattern starts at the left side of rectangular parallelepiped divider 124 and ends at the right side of upper half 121 so as to create an upper semicircular space inside said upper half above divider 124. In a similar fashion, second plurality of sections 122_1 further comprises four sections. Second zigzag pattern starts at the left side of rectangular parallelepiped divider 124 and ends at the right side of lower half 122 so as to create a lower semicircular space inside lower half 122 below divider rectangular parallelepiped divider 124.

Back heel portion 135 further comprises first spring 1351 parallel to second spring 1352, each having a zigzag shape.

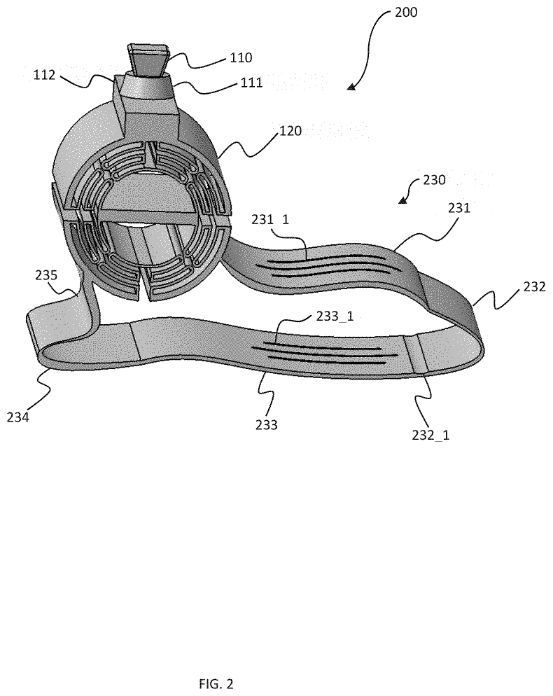

Now referring to FIG. 2, a second implementation of a prosthetic foot 200 in accordance with an exemplary embodiment of the present invention is illustrated. Prosthetic foot 200 also includes same socket assembly 110, sideway cylindrical ankle joint 120 and a foot assembly 230 laterally affixed to sideway cylindrical ankle joint 120. In many implementations of the present invention, foot assembly 230 further comprises a dorsal portion 231, a phalange (or toe) portion 232, a sole portion 233, and a heel portion 230. In a more detailed aspects of the second implementation, dorsal portion 230 has only one arch membrane with longitudinal parallel slits 230_1. Sole portion 232 also has parallel longitudinal (lengthwise) slits 232_1. Finally, sole portion 234 includes one sheet with an arch back sole portion 235 that connects to sole portion 234 and sideway cylindrical ankle joint 130 respectively.

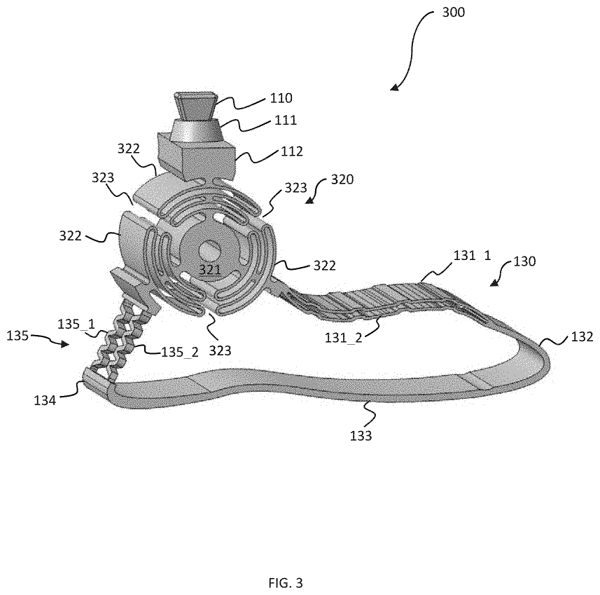

Next referring to FIG. 3, a third implementation of a prosthetic foot 300 in accordance with an exemplary embodiment of the presentation is illustrated. Prosthetic foot 300 also includes same socket assembly 110, a sideway cylindrical ankle joint 320 and a foot assembly 130 laterally affixed to sideway cylindrical ankle joint 320. In many implementations of the present invention, foot assembly 130 is the same as that described in FIG. 1 and further comprises dorsal portion 131, phalange (or toe) portion 132, ole portion 133, heel portion 134, and back heel portion 135. As the reference numbers indicate, the only difference between the first implementation and the third implementation is sideway cylindrical ankle joint 320. Sideway cylindrical ankle joint 320 is constituted of a hollow cylindrical core 321 radially connected with three different zig zag sections 322 respectively. In that regard, the circumference of hollow cylindrical core 321 is connected to three different zig zag sections 322 that have gaps 323 between them.

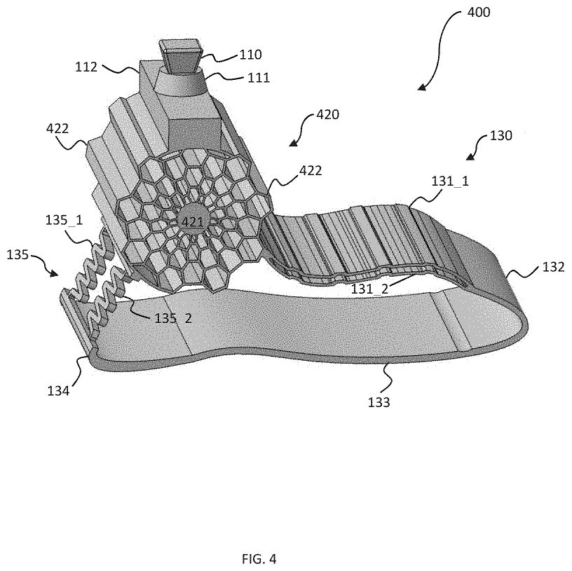

Next referring to FIG. 4, a fourth implementation of a prosthetic foot 400 in accordance with an exemplary embodiment of the presentation is illustrated. Prosthetic foot 400 also includes same socket assembly 110, a sideway cylindrical ankle joint 420 and foot assembly 130 laterally affixed to sideway cylindrical ankle joint 420. In many implementations of the present invention, foot assembly 130 further comprises dorsal portion 131, phalange (or toe) portion 132, sole portion 133, a heel portion 134, and a back heel portion 135 as described in FIG. 1. As the reference numbers indicate, the only difference between the first implementation and the fourth implementation is at sideway cylindrical ankle joint 420. Sideway cylindrical ankle joint 420 is constituted of a solid cylindrical core 421 and a plurality of hollow polygon tubes 422 connected to the circumference of and arranged radially from solid cylindrical core 421. In one detailed aspect of the present invention, each hollow polygon tub 422 has different surface areas so that sideway cylindrical ankle joint 420 resembles a beehives structure.

Referring again to FIG. 1-FIG. 4, in operation, the walking gait of prosthetic feet 100-400 is a continual series of steps that constitutes a rhythm of a swing phase and a support phase. In the swing phase, prosthetic foot 100-400 swings in the air and the natural foot is in on the ground to provide support. In the support phase prosthetic foot 100-400 is on the ground to provide support while the other natural foot swings in the air. The two phases alternatively repeats during the walking cycle to create the rhythm. The reason the patient can walk forward is that two feet alternatively change between swing phase and support phase. During the walking cycle, there is no moment when both feet are up in the air and not touching the ground.

The support phase is the time when prosthetic foot 100-400 touches the ground and when sole portion 133 lifts out of the ground. The support phase is further divided into the time when heel portion 134 is off ground, sole portion 133 is flat to the ground, arch 133_2 touches the ground, heel portion 134 off the ground and pre-swing period (when phalange portion 132 off the ground).

In the swing phase, this phase starts when sole portion 133 lifts off from the ground to the time it touches the ground again. This phase is further divided into pre-swing, mid-swing, and terminal swing. Moreover, in sideway cylindrical ankle joint 120, 320, or 420 (the tibia and fibula bone right above the ankle) provides an angular momentum when dorsal portion 131 or 231 (the metatarsal) has a short fold in the beginning of the support phase when sole portion 133 or 233 first touches the ground. The transition to the folding momentum of the sole portion 133 or 233 to control the rotation of the leg upon prosthetic foot 100-400. Afterward the momentum continues to fold sole portion 133 or 233 when the design and the compliant composite material in foot portion 130-430 contracts at the center of gravity to propel prosthetic foot 100-400 forward. At the beginning of this phase, prosthetic foot 100-400 swings, the folding of sole portion 133 or 233 continues due to the implementations described above in accordance with the present invention. Then sole portion 133 or 233 contracts at the center of the gravity. In the mid-swing, prosthetic foot 100-400 swings there is a little force felt at the calf bone.

The support phase is the time when prosthetic foot 100-400 is on the ground. It comprises about 60% of the walking cycle. For part of the support phase, both feet will be on the ground for a period of time. The support phase is further divided into five sub-stages that prosthetic foot 100-400 undergoes. They are as follows. Heel strike, early flatfoot, late flatfoot, Heel rise, and toe off.

The heel strike phase starts the moment when heel portion 134 first touches the ground, and lasts until prosthetic foot 100-400 is on the ground (early flatfoot stage).

The beginning of the “early flatfoot” stage is defined as the moment that the whole prosthetic foot 100-400 is on the ground. The end of the “early flatfoot” stage occurs when the body's center of gravity passes over top of prosthetic foot 100-400. The body's center of gravity is located approximately in the pelvic area in front of the lower spine, when a patient (not shown) stands and walks. The main purpose of the “early flatfoot” stage is to allow prosthetic foot 100-400 to serve as a shock absorber, helping to cushion the force of the body weight landing on prosthetic foot 100-400.

In the late flatfoot stage: once the body's center of gravity has passed in front of the neutral position, the late flatfoot stage occurs. The “late flatfoot” stage of gait ends when heel portion 134 lifts off the ground. During the “late flatfoot” phase of gait, prosthetic foot 100-400 needs to go from being a flexible shock absorber to being a rigid lever that can serve to propel the body forward.

In the heel raise phase: as the name suggests, heel portion 134 rise phase begins when heel portion 134 begins to leave the ground. During this phase, the foot functions as a rigid lever to move the body forward. During this phase of walking, the forces that go through prosthetic foot 100-400 are quite significant: often 2-3x a person's body weight. This is because prosthetic foot 100-400 creates a lever arm (centered on the ankle), which serves to magnify body weight forces. Given these high forces and considering that the average human takes 3000-5000 steps per day (an active person commonly takes 10,000 steps/day). The implementations of prosthetic foot 100-400 enable the user to avoid chronic repetitive stress-related problems, such as metatarsalgia, bunions, posterior tibial tendon dysfunction, peroneal tendonitis, and sesamoiditis.

Finally, the toe off stage of gait begins as phalange portion 132 leave the ground. This represents the start of the swing phase.

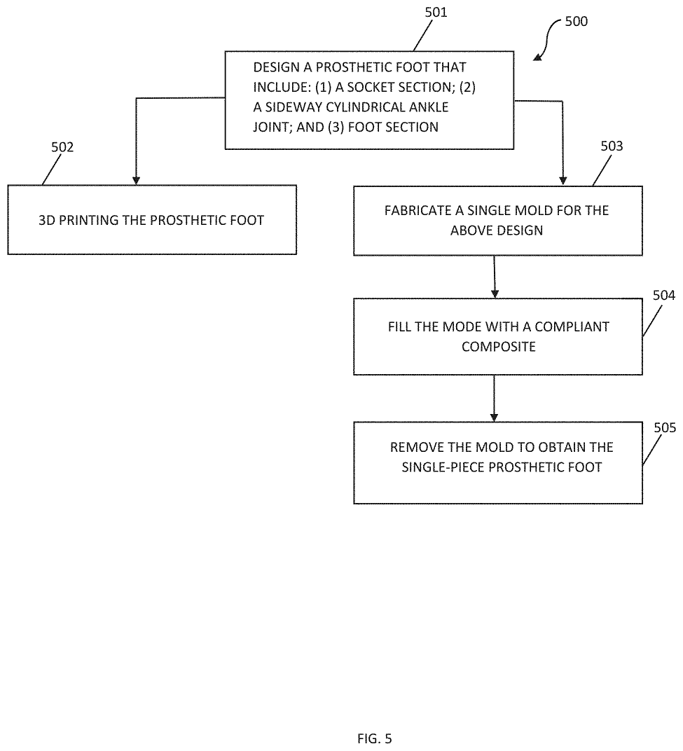

Now referring to FIG. 5, a method 500 of fabricating prosthetic foot 100-400 described above is illustrated. In many implementations of the present invention, method 500 aims to produce a single integral piece prosthetic foot made up of a compliant composite that includes the steps of providing a mold that includes a socket section, a sideway cylindrical section, and a foot section.

At step 501, a prosthetic foot comprised of a socket section, a sideway cylindrical ankle joint having a maze-like structure, and a foot section is designed.

By way of specific examples, as shown in FIG. 1-FIG. 4, sideway section, cylindrical ankle joint, and foot section as described in details above is designed using a computer-assisted design (CAD) program such as Solid Works. In practice, different and other 3D design software such as Onshape, Netfabb, fusion 360, Craftware, etc. can be used to design prosthetic foot 100-400.

At step 502, after the design is completed, it is printed out using a 3D printer. In many implementations of step 502, 3D printing software can be used to print the entire prosthetic foot 100-400 in a single integral piece using Glass Fiber Reinforced Plastic (GFRP).

Alternatively, at step 503, a single integral mold is fabricated using the design of step 501.

Next, at step 504, the mold is filled with a compliant composite material. Step 502 is implemented using a such as Glass Fiber Reinforced Plastic (GFRP).

Finally, at step 505, the mold is removed. In implementation, the mold is removed to achieve prosthetic foot 100-400 as described in FIG. 1-FIG. 4.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

### DESCRIPTION OF NUMERALS

100 first embodiment of the prosthetic foot
110 socket assembly
111 base portion
112 extension
120 sideway cylindrical ankle joint
121 upper half
121_1 upper section having zig-zag structure
122 lower half
122_1 lower section having zig-zag structure
123_1 left gap
123_2 right gap
124 divider
130 dorsal portion
131_1 upper membrane of the dorsal portion
131_2 lower membrane of the dorsal portion
132 phalange portion
132_1 first end of the phalange portion
132_2 second end
133 sole portion
133_1 groove
133_2 arch in the sole portion
135 back heel portion
135_1 first zig-zag back heel portion
135_2 second zig-zag back heel portion
200 second embodiment of the prosthetic foot
230 dorsal portion in the second embodiment
231 single dorsal membrane
231_1 parallel longitudinal slits on single dorsal membrane
232 phalange portion in the second embodiment
233 sole portion in the second embodiment
233_1 parallel longitudinal slits on the sole portion
234 heel portion
234_1 arch shaped back heel portion
300 third embodiment of prosthetic foot
320 sideway cylindrical ankle joint
321 hollow cylindrical core of the sideway cylindrical ankle joint
322 sections with zig-zag structure
323 gaps between sections with zig-zag structure
400 fourth embodiment of the prosthetic foot
420 sideway cylindrical foot ankle joint
421 solid cylindrical core
422 polygon tubes

1. A self-watering modular planter, comprising:
a plurality of modular trays configured to provide a growing medium, each of said modular tray having an open top side, a bottom side, a left side, a right side, a front side, and a back side; and
an extendable frame skeleton connected to secure said plurality of modular trays; wherein:
a space inside said modular tray further comprises:
a first bar member welded to said left side and said right side spanning across the length of said modular tray;
a second bar member, welded to said left side and said right side spanning across the length of said modular tray, disposed parallel to said first plate;
a plurality of dividers welded to said front side and said back side of said modular tray and perpendicular to said first bar member and said second bar member;
a plurality of receptors disposed on said bottom side, configured to provide means for said extendable frame skeleton to insert therethrough;
a bottom surface vertically dividing said modular tray into a water tank and said growing medium;
an array of capillary tubes, disposed along said bottom surface and in fluid communication with said water tank, operable to provide water to soils of said modular tray by means of a capillary action; and
said left side and said right side further comprises a first connector and a second connector respectively configured to connect to other of said modular trays;
a set of legs, arranged at four corners of said bottom, configured to slide snugly to said first bar member and said second bar member when said plurality of modular trays are stacked vertically.

2. The self-watering modular planter tower claim 1 wherein said water tank further comprises outlets disposed on said left side and said right side of said modular tray.

3. The self-watering modular planter tower claim 1 wherein extendable frame skeleton further comprises:
a plurality of vertical U-shaped frames having a first series of adjusting holes disposed along the length of said vertical U-shaped frames and a first adjusting locking mechanism;
a plurality of horizontal auxiliary tubes having a second series of adjusting holes disposed along the length of said horizontal auxiliary tubes and a second set of adjusting locking mechanism, wherein
the length of said plurality of said vertical U-shaped tubes is extended by inserting said plurality of horizontal auxiliary tubes at either end so as said first series of adjusting holes is lined up with said second series of adjusting holes; and
a plurality of horizontal the length of said plurality of tubes is extended by inserting other tube to either ends so as said second series of adjusting holes are lined up.

4. The self-watering modular planter tower claim 1 further comprises a mat disposed on said bottom surface of said modular tray.

5. The self-watering modular planter tower claim 4 wherein said mat is a capillary mat having a plurality of layers capable of absorbing and releasing water by the capillary action.

6. The self-watering modular planter tower claim 4 wherein said mat further comprises an array of drainage holes disposed throughout an area of said mat.

7. The self-watering modular planter tower claim 5 wherein said mat further comprises a thin sheet of sponge capable of absorbing and releasing water.

8. The self-water modular planter tower of claim 6 wherein each of said capillary tubes further comprises:
a protecting outer shelf firmly connected to said bottom of said modular tray; and
a capillary material inserted inside said protecting outer shelf and in fluid communication with said water tank.

9. The self-water modular planter tower of claim 7 wherein said protecting outer shelf comprises a cylindrical tube and said capillary material comprises a cloth.

10. The self-water modular planter tower of claim 7 wherein said capillary material comprises a fiber capable of drawing water from said water tank to soils filled inside said growing medium.

11. The self-water modular planter tower of claim 7 said left side and said right side each has a fan shape.

12. A method of manufacturing a self-watering modular planter assembly, comprising:
providing a vertical N by M growing medium comprising a plurality of modular trays capable of securely connecting to one another, each of said modular tray having an open top side, a bottom side, a left side, a right side, a front side, and a back side, wherein said bottom side of said modular tray is vertically divided into a water tank and a growing medium;
providing an extendable frame skeleton capable of inserting into each of said plurality of modular trays so as to secure said plurality of modular trays;
providing an array of capillary tubes, disposed vertically along said bottom side and in fluid communication with said water tank, operable to provide water to a soil of said modular tray by means of capillary action; and
providing a capillary mat disposed inside each of said modular tray.

13. The method of claim 12 wherein a space inside said modular tray further comprises:
a first bar member welded to said left side and said right side spanning across the length of said modular tray;
a second bar member, welded to said left side and said right side spanning across the length of said modular tray, disposed parallel to said first plate;
a plurality of dividers welded to said front side and said back side of said modular tray and perpendicular to said first bar member and said second bar member;
a plurality of receptors disposed on said bottom side, configured to provide means for said extendable frame skeleton to insert therethrough;
a bottom surface vertically divided said modular tray into said growing medium and said water tank;
a set of legs, arranged at four corners of said bottom side, configured to connected to said first bar member and said second bar member when said plurality of modular trays are stacked vertically into said vertical N by M growing medium; and
said water tank further comprising outlets disposed on said left side and said right side of said modular tray.

14. The method of claim 12 wherein said providing an extendable frame skeleton further comprises:
providing a plurality of vertical U-shaped frames having a first series of adjusting holes disposed along the length of said vertical U-shaped frames and first adjusting locking mechanism;
providing a plurality of auxiliary tubes having a second series of adjusting holes disposed along the length of said tubes and a second set of adjusting locking mechanism, wherein:
the length of said plurality of said vertical U-shaped tubes is extended by inserting said plurality of auxiliary tubes at either end so as said first series of adjusting holes is lined up with said second series of adjusting holes; and
providing a plurality of extendable horizontal tubes, connected to said plurality of U-shaped frames in a direction parallel to the length of said modular tray defined by said front side and said back side, wherein
the length of said plurality of extendable horizontal tubes is extended by inserting said auxiliary tubes to either ends thereto.

15. The method of claim 12 wherein said capillary mat further comprises an array of drainage holes disposed throughout a surface area of said mat.

16. The method of claim 12 wherein said mat further comprises a thin sheet of sponge capable of absorbing and releasing water.

17. The method of claim 12 wherein each of said capillary tubes further comprises:
a protecting outer shelf firmly connected to said bottom of said modular tray;
a capillary material inserted inside said protecting shelf and in fluid communication with said water tank.

18. The method of claim 14 wherein said protecting shelf comprises a cylindrical tube and said capillary material comprises a cloth.

19. A method of growing plants in a limited space area, comprises:
providing a plurality of modular trays, each of said modular tray having an open top side, a bottom, a left side, a right side, a front side, and a back side, wherein said bottom side of said modular tray further comprises a water tank;
providing an extendable frame skeleton capable of inserting into each of said plurality of modular trays so as to secure said plurality of modular trays;
providing an array of capillary tubes, disposed vertically along said bottom side and in fluid communication with said water tank, operable to provide water to a soil of said modular tray by means of capillary action;
providing a plurality of capillary mats each capable of absorbing and releasing water;
providing male and female locking means for securely interlocking said plurality of modular trays together;
laying each of said capillary mats on a bottom surface deposited on top of said water tank from;
assembling said plurality of modular trays and said extendable frame skeleton to form a vertical N by M array of growing medium;
filling each of said modular trays with soil;
filling said water tank with water; and
growing plants in each of said plurality of modular trays.

20. The method of claim 19 wherein each of said capillary tubes further comprises:
a protecting outer shelf firmly connected to said bottom surface of said modular tray; and
a capillary material inserted inside said protecting shelf and in fluid communication with said water tank.
